(19) United States
(12) Patent Application Publication
(10) Pub. No.: US 2015/0366668 A1
(43) Pub. Date: Dec. 24, 2015

(54) CELLULAR-SCALE SURFACE MODIFICATION FOR INCREASED OSTEOGENIC PROTEIN EXPRESSION

(71) Applicant: COMMUNITY BLOOD CENTER, Dayton, OH (US)

(72) Inventors: Shawn A. Hunter, Springboro, OH (US); Mary E. Blackmore, Dayton, OH (US); Felicia C.T. Gooden, Beavercreek, OH (US); Cody W. Saylor, Huber Heights, OH (US)

(73) Assignee: COMMUNITY BLOOD CENTER, Dayton, OH (US)

(21) Appl. No.: 14/747,244

(22) Filed: Jun. 23, 2015

Related U.S. Application Data
(60) Provisional application No. 62/015,680, filed on Jun. 23, 2014.

(51) Int. Cl. A61F 2/28

(52) U.S. Cl. CPC A61F 2/28; A61F 2002/2835; A61F 2002/2867; A61F 2310/00359

(57) ABSTRACT

According to various embodiments, a bone product includes bone having at least one patterned surface. The patterned surface includes a plurality of pits. Each of the plurality of pits has a diameter of from about 5 μm to about 15 μm and a depth of approximately half of the diameter.

### CROSS-REFERENCE TO RELATED APPLICATIONS

This patent application claims the benefit of Provisional Patent Application Ser. No. 62/015,680 filed on Jun. 23, 2014, which is hereby incorporated by reference in its entirety.

### TECHNICAL FIELD

The present specification generally relates to bone products and, more specifically, to allograft bone products including surface modification to increase osteogenic protein expression.

### BACKGROUND

The bone remodeling process relies on cellular adhesion and cell signaling. In particular, good cellular adhesion can permit better incorporation of graft material through positive host cell response and infiltration. Cell signaling is crucial in determining how a cell and the tissue it develops will react and work within its environment. Various studies have determined that environmental modifications can be effective in adjusting and/or controlling cellular differentiation and activity.

Cellular differentiation and activity can be controlled via the environment through any of three main routes: material chemistry, material stiffness, and material/surface topography. Orthopedic devices and/or materials that possess engineered (or synthetic) surfaces with micro-features have consistently been shown to influence cellular function, response and adhesion capabilities. At the nanoscale, bone tissue contains a complex mixture of topographical pits, protrusions and fibers. These all arise from the mineral-collagen composite that constitutes mineralized osteoid. While many studies have attempted to mimic this complex mixture, the substrates have demonstrated a decrease in focal adhesion formation relative to the controls of the studies, which in turn decreases a cell's ability to interact with its environment.

Other studies have explored development of nanofeatures or micro-features on various engineered surfaces, such as plastics, polymers, epoxies, and metals such as titanium, cobalt, and chromium.

Despite the efficacy of previous studies and research, there are some significant drawbacks to these approaches. Conventional approaches incorporate fabrication methods that do not generate features on a small enough scale to be recognized and/or beneficial to osteoblastic growth and adhesion. Additionally, conventional methods are focusing on engineered rather than natural materials. With respect to long term tissue regeneration solutions, natural materials, already designed to be utilized within the body, demonstrate more promise especially in the realm of biocompatibility.

Accordingly, a need exists for natural biomaterials such as bone that include features that can improve cellular adhesion and, in turn, improve osteoinductive healing potential and biocompatibility of orthopedic implants.

### SUMMARY

According to one embodiment, a bone product includes bone having at least one patterned surface. The patterned surface includes a plurality of pits. Each of the plurality of pits has a diameter of from about 5 μm to about 15 μm and a depth of approximately half of the diameter.

According to another embodiment, a bone product includes cortical bone having at least one patterned surface. The patterned surface includes a plurality of pits. Each of the plurality of pits has a diameter from about 5 μm to about 15 μm and a pitch of about 1.5 to about 2.5 times the diameter of each pit, as measured from the center of one pit to the center of an adjacent pit.

According to still another embodiment, an allograft bone includes at least one surface modification. The surface modification includes a plurality of pits, each of the plurality of pits having a diameter of from about 5 μm to about 15 μm.

Additional features and advantages will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 schematically depicts a bone product in the form of a bone block in accordance with one or more embodiments described herein;

FIG. 2 schematically depicts a 48-well plate map for use in one or more embodiments described herein;

FIG. 3 is a graph illustrating autofluorescence of bone based on wavelength according to one or more embodiments described herein;

FIG. 4 is a graph illustrating change in BMP2 over time within the same sample type according to one or more embodiments described herein;

FIG. 5 is a graph illustrating change in BMP8a over time within the same sample type according to one or more embodiments described herein;

FIG. 6 includes photographs depicting surface roughness of control and patterned samples according to one or more embodiments described herein;

FIG. 7 is a graph illustrating change in surface roughness as a percentage over time according to one or more embodiments described herein;

FIG. 8 is a graph illustrating change in average pit diameter over time according to one or more embodiments described herein;

FIG. 9 is a graph illustrating change in average pit volume over time according to one or more embodiments described herein;

FIG. 10 is a graph illustrating change in average pit depth over time according to one or more embodiments described herein;

FIG. 11 is a graph illustrating change in average bone area over time according to one or more embodiments described herein;

FIG. 12 includes photographs depicting bone resorption and bone formation over time according to one or more embodiments described herein;

FIG. 13 is a graph illustrating overall surface roughness in blocks treated with BMSCs, FBs, and ASCs according to one or more embodiments described herein;

FIG. 14 is a graph illustrating overall pit diameter and depth in blocks treated with BMSCs, FBs, and ASCs according to one or more embodiments described herein;

FIG. 15 is a graph illustrating overall pit volume in blocks treated with BMSCs, FBs, and ASCs according to one or more embodiments described herein;

FIG. 16 is a graph illustrating change in net bone area over time in bone treated with ASCs according to one or more embodiments described herein;

FIG. 17 is a graph illustrating change in net bone area over time in bone treated with FBs according to one or more embodiments described herein;

FIG. 18 is a graph illustrating change in net bone area over time in bone treated with BMSCs according to one or more embodiments described herein;

FIG. 19 is a graph illustrating overall DNA content in area at 4 weeks and 8 weeks according to one or more embodiments described herein; and

FIG. 20 is a graph illustrating tissue area of cortical bone, cartilage, and trabecular bone at 8 weeks according to one or more embodiments described herein.

### DETAILED DESCRIPTION

Reference will now be made in detail to various embodiments of bone products including at least one patterned surface. The patterned surface includes a plurality of pits, which may generally have diameters of from about 5 μm to about 15 μm and depths of about half of the diameter. In various embodiments, the pits are spaced apart such that the pits have a pitch of about 1.5 to about 2.5 times the diameter of each pit, as measured from the center of one pit to the center of an adjacent pit. Accordingly, when the bone product is incorporated into a host, improved osteoinductive healing potential and biocompatibility may be observed.

Bone tissue regeneration, also referred to as fracture healing, is a proliferating physiological process in which the body's necessary cellular components facilitate the repair of any form of bone damage. There are several steps involved in this process, all facilitating the protection and rejuvenation of the areas surrounding the damaged tissue. The length of the process is dependent upon the extent of the damage or injury in addition to a person's healing potential. Additionally, the angle of dislocation or fracture can also play a crucial role in healing/regeneration time.

Regardless of the finer details, all bone healing efforts involve three distinct phases: (i) the reacting phase, (ii) the reparative phase and (iii) the remodeling phase. Once trauma or injury occurs, the reacting phase begins. It simply involves the body “reacting” to the injury or problem through a series of protective actions. Initially, blood cells within the tissue adjacent to the injury site make themselves known, while nearby blood vessels work to constrict blood flow and prevent any further bleeding. The blood cells then proceed to form a blood clot. These blood cells, and those in the immediate environment of the clot, degenerate and die, and are survived by the surrounding fibroblasts. The fibroblasts then form a loose aggregate of cells, which becomes the foundation for the beginnings of preliminary tissue.

The reparative phase usually occurs days after the fracture or injury, and involves the development of collagen matrix and lamellar bone. The lamellar bone (woven bone) is formed from osteoblasts that have travelled via vascular channels to the repair site. They penetrate the area and form the precursor to trabecular bone, restoring the majority of the bone's original strength.

The final phase of regeneration and healing is the remodeling phase, which is also the phase where the majority of this research is focused. It is during this phase that compact bone is substituted for the previously developed trabecular (cancellous) bone. The trabecular bone is first resorbed by osteoclasts (cells that break down and absorb bone), creating a shallow pit. Osteoblasts then develop and deposit all the necessary components of compact bone including osteoid, mineralized matrix, pertinent enzymes and proteins.

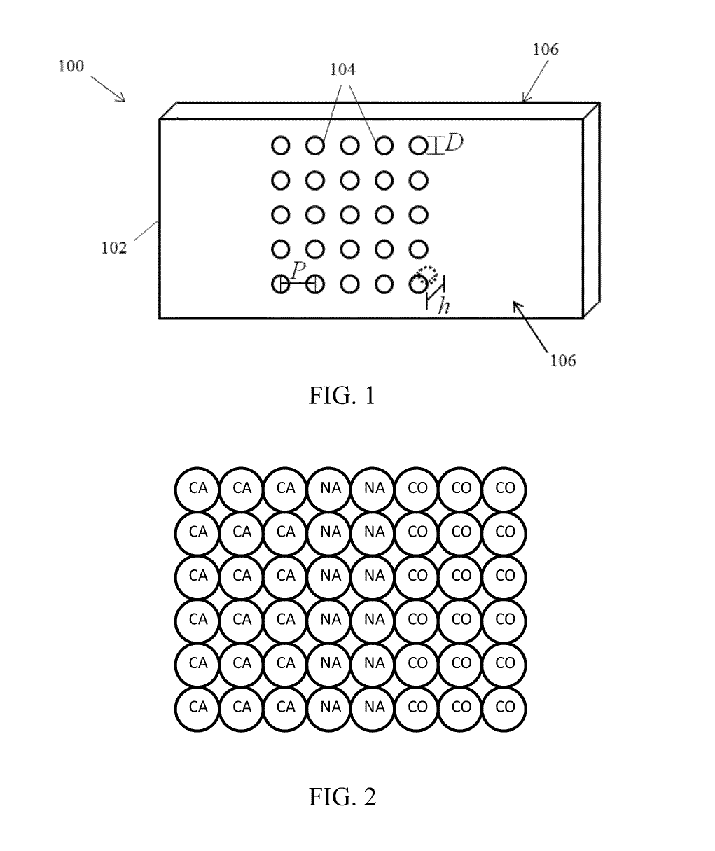

FIG. 1 depicts a bone product 100 in the form of a bone block 102. The bone block 102 includes a patterned surface in the form of a plurality of pits 104. The fabrication of the pattern increases the surface roughness on the bone and increases the overall surface area, which in turn gives cells a greater opportunity to adhere and permits more cells to take part in bone regeneration. By increasing the number of cells participating in bone regeneration, healing times may be decreased. Moreover, studies have shown that surfaces with rougher topographies have demonstrated that they help cells promote byproducts more conducive to long term adhesion stability.

In various embodiments, such as the embodiment depicted in FIG. 1, the pits 104 are arranged in a matrix, although other arrangements may be used depending on the particular embodiment. Each of the pits 104 has a diameter D and a depth h. Each of the pits 104 may be hemispherical in shape, with the walls of the pit sloping downward and inward from the top of the pit. In various embodiments, the pit has a curved bottom. In some embodiments, the pit may have a conical bottom. In some embodiments, each pit may be cylindrical in shape, such that the diameter of the pit at the top and the diameter of the pit at the bottom are substantially equal to one another. Other feature shapes may be employed, although pits or other circular features are preferred as various studies have shown that square and hexagonal features may result in negligible cell spreading and elongated cell morphology.

In various embodiments, the diameter D of each pit is from about 5 μm to about 15 μm. In some embodiments, each of the plurality of pits 104 has a diameter of from about 7 μm to about 12 μm. In still other embodiments, each of the plurality of pits 104 has a diameter of about 10 μm. In various embodiments, pits are sized to have a diameter of greater than or equal to about 5 μm to enable cells to enter the pits, as smaller pits may be too small for cells to get in to. Additionally, pits smaller than about 5 μm in diameter may be difficult to form in bone due to the inherent roughness of the bone. Pits greater than about 15 μm in diameter may result in isolation of cell groups and limited cell interaction.

In various embodiments, each pit 104 has a depth h that is approximately half of the diameter D. Accordingly, in various embodiments, each pit 104 has a depth h of from about 2.5 μm to about 7.5 μm. In some embodiments, each of the plurality of pits 104 has a depth of from about 3.5 μm to about 6 μm. In still other embodiments, each of the plurality of pits 104 has a depth of about 5 μm. The depth h may vary, but should generally be deep enough to increase surface area without being so deep that the cells become trapped in the pit.

The plurality of pits 104 are spaced apart from one another such that a pit pitch as measured from the center of one pit to the center of an adjacent pit is approximately about 1.5 to about 2.5 times the diameter of each pit. In some embodiments, the pitch is between about 7.5 μm and about 37.5 μm. In some embodiments, the pitch is approximately 20 μm. Although other pitches may be selected, a pitch that is too great (e.g., greater than about 20 μm) may prevent cells from interacting with one another and may interrupt cell signaling. Similarly, a pitch that is too small may simply result in a roughened surface of the bone block 102 without increasing the surface area. In various embodiments, the pits increase the surface roughness by about 35% to about 105%. In some embodiments, the pits increase the surface roughness by about 45% to about 80%. In still other embodiments, the pits increase the surface roughness by about 70%.

According to various embodiments, the bone block 102 is formed from cortical bone. However, in some embodiments, the bone block 102 may be formed from cancellous bone. In still other embodiments, the bone block 102 may include a combination of cortical and cancellous bone. In various embodiments, the bone is an allograft bone (i.e., a human bone) or a xenograft bone. The human bone may be, for example, from a cadaver or from a living donor. Xenograft bones may be, for example, porcine, ovine, or bovine.

In various embodiments, the patterned surface is laser-machined into the bone. The use of a laser enables the formation of pits to be highly repeatable and enables the dimensions of the pits to be finely selected. Additionally, the non-contact laser machining process minimizes handling of the bone, which can decrease the exposure to and risk of environmental contamination. Laser machining is also non-invasive to the structural integrity of the bone.

Prior to laser machining the bone, in various embodiments, the major faces 106 of the bone block 102 are end-milled to create relatively smooth and parallel surfaces. The end-milling may be performed using a computer numerical control (CNC) machine.

In various embodiments, the bone is laser machined using an ultrashort pulse (USP) laser. As used herein, an ultrashort pulse laser is a laser that produces a laser pulse with a duration of 100 picoseconds (ps) or less. Other lasers may be utilized, provided they are able to ablate material, such as bone, with little transfer of thermal energy to the material. Once an appropriate laser is chosen, in various embodiments, an optical path to manipulate and focus the laser on a target workspace is assembled and a firing pattern to produce the desired texture is designed and coded into a control system for the laser. The beam path includes elements for beam expansion and focusing. The control system can govern the pulse frequency, the rate at which the laser beam moves across the target surface (i.e., mark speed), and implement a motion pattern for the beam that is defined by a pattern file.

According to various embodiments, the pulse duration of the laser is less than about 100 ps. In some embodiments, the pulse duration of the laser is less than about 50 ps. In still other embodiments, the pulse duration of the laser is less than about 20 ps. In various embodiments, the wavelength of the laser may be from about 300 nm to about 1200 nm, although the wavelength may be any suitable wavelength outside of this range in some embodiments.

In various embodiments, the laser has a pulse repetition frequency of from about 75 kHZ to about 250 kHz. In some embodiments, the pulse repetition frequency is from about 100 kHZ to about 200 kHz. In still other embodiments, the pulse repetition frequency may vary provided it gives a suitable combination of pulse overlap and pulse energy. According to various embodiments, the laser has an effective laser spot size of from about 10 μm to about 20 μm. The effective laser spot size may be determined, for example, by the measurement of realized texture dimensions. According to various embodiments, the laser has a depth per pass of from about 2.5 μm to about 10 μm, and the average power is from about 200 mW to about 300 mW.

In one particular embodiment, the bone may be laser machined using a Lumera SuperRAPID laser having a pulse duration of 12 ps, a wavelength of 355 nm, and a pulse repetition frequency of 200 kHz. The effective laser spot size is about 17 μm, the depth per pass is about 2.5 μm, and the average power is about 200 mW. In another embodiment, the bone may be laser machined using a JenLas D2.FS laser having a pulse duration of about 0.3 ps, a wavelength of 1025 nm, and a pulse repetition frequency of 100 kHz. The effective laser spot size is about 10 μm, the depth per pass is about 10 μm, and the average power is about 280 mW.

After machining, in various embodiments, the bone block 102 may be subjected to terminal sterilization. Terminal sterilization may be, for example, by γ-irradiation, electron beam sterilization, or other known terminal sterilization techniques.

According to various embodiments, the bone product 100 may be used in a transplant. For transplant, the bone product 100 may be in the form of a bone block, such as is shown in FIG. 1, or may be in the form of a pin, dowel, strip, strut, wedge, or the like. The patterned surface of the bone product 100 may be a flat surface or a curved surface. In some embodiments, the bone product may be in the form of particulate bone. In embodiments in which the bone product 100 is in the form other than a bone block, it should be understood that the bone can be formed into the appropriate shape or form prior to laser machining the patterned surface onto one or more faces or surfaces of the bone product.

In various embodiments, prior to using the bone product in a transplant (e.g., before insertion into a host environment), the bone product may be seeded with stem cells or primary cells, while in other embodiments, the bone product is not seeded with cells. For example, adipose-derived stem cells (ASCs) or bone marrow-derived stem cells (BMSCs) may be seeded onto the bone product. As another example, primary cell lines, such as fibroblasts, osteoblasts, and osteoclasts may be seeded onto the bone product. Scaffolds seeded with cells may provide increased potential for accelerated healing. ASCs are abundant and may be easily derived from human adipose post liposuction. Additionally, ASCs have the ability to differentiate along multiple lineage pathways, making them a model for tissue engineering applications. BMSCs are the progenitor cells for skeletal tissues (e.g., are active in bone remodeling) and are likely to be the first responders to an implant site.

The bone products described herein may be used in bone transplants or in other surgeries such as dental or oral surgery, joint reconstruction, spinal fusion, bone and joint repair, or the like. As described herein, the bone product may be implanted into a host patient and may provide improved osteoinductive healing potential and implant integration as compared to implants without patterned surfaces or incorporating materials other than bone.

In order that various embodiments may be more readily understood, reference is made to the following examples which are intended to illustrate various embodiments, but not limit the scope thereof.

### EXAMPLES

In the various examples provided hereinbelow, certain experimental procedures were followed. Various examples make reference to the following experimental procedures:

### Cell Culture

Primary human and pluripotent human stem cell lines were cultured and passaged to bank for future applications. The cells were initially purchased in a cryopreserved state, at a concentration of greater than or equal to 500,000 cells/mL, with 1 mL purchased. These cells were immediately plated and cultured. Once the cells reached greater than 80% confluence, they were lifted from their current culture dish and replated in new culture dishes to account for the increase in cell population and proliferation (i.e., passaging). These cells were passaged three times to bring them into a mature cell age. Cells were then cryogenically preserved for future use.

Adequate amounts of both primary human osteoblasts and adipose derived adult stem cells were removed from the cryotank for various examples. The tubes were placed immediately in a 37° C. water bath for 90 seconds with gentle agitation to thaw. Once cells were completely thawed, the cryotubes were sprayed with 70% alcohol and placed in sterile hood. Contents of all tubes were combined with 5 mL of prewarmed media and placed in 15 mL conical tubes. The cell/media suspension was centrifuged at 300×g for 5 minutes at room temperature. Tubes were then removed from the centrifuge and sprayed with 70% alcohol and placed in hood. The supernatant was aspirated from each tube being careful not to disturb each cell pellet. An adequate amount of fresh media was placed in each tube and the cells were resuspended within the media. The cells were then counted using a disposable hemocytometer to get an estimate of cell density per milliliter of media. This helps formulate the amount of cell-media suspension we will need to place on each bone sample and maintain a consistent seeding density.

### Manual & Automated Cell Counting

The standard method for counting cells involves the use of a hemocytometer. It consists of a thick glass microscope slide with a rectangular indentation that creates a chamber. This chamber is engraved with a laser-etched grid of perpendicular lines. The device is carefully crafted so that the area bounded by the lines is known, and the depth of the chamber is also known. It is therefore possible to count the number of cells or particles in a specific volume of fluid, and thereby calculate the concentration of cells in the fluid overall.

For all manual cell counts, each of the circled regions (A-D) as well as the center section of counting squares were counted and averaged. The cells per ml were then calculated using the following:

$$\frac{Cells}{mL} = ( Average Cell Count ) ( Dilution Factor ) ( {10}^{4} )$$

Newer, more recent technologies permit automated cell counting via a handheld device. The Sceptor 2.0 (Millipore, CAT PHCC20040) provides a fast and convenient method for counting cells and particles. The user prepares a dilution of the cell culture of interest and uses the Scepter cell counter to aspirate a sample of this dilution into the Scepter sensor. It helps reduce the variability associated with manual counting and was used to help validate the accuracy of the manual cell counts associated with this experimental series.

### Automated Western Blotting

A Simple Western is an automated Western—no gels, no transfer devices, no blots, no film and no manual analysis. Samples are loaded in the automated system and start is pressed. The automated system automates all steps of the process including sample loading, size-based protein separation, immunoprobing, washing, detection and data analysis for up to 12 samples simultaneously. Simple Western assays take place in a capillary. Samples are treated with sodium dodecyl sulfate (SDS) and dithiothreitol (DTT) and then heat denatured. Assay reagents and prepared samples are loaded into an assay plate and placed in the automated system. The automated system loads samples into the capillary automatically. Proteins are separated by size as they migrate through both a stacking and separation matrix. The separated proteins are then immobilized to the capillary wall via a proprietary, UV capture method. Target proteins are identified using a primary antibody and immunoprobed using an HRP-conjugated secondary antibody and chemiluminescent substrate. The resulting chemiluminescent signal is detected and quantitated. Analysis is automatically performed and results are presented in Compass software.

### Example 1

The primary objective of this example was to identify micron-scale surface characteristics that promote primary and progenitor cell viability and proliferation, while upregulating osteogenic proteins. Human osteoblasts (OBs) and adipose-derived stem cells (ASCs) were cultured up to 21 days on chambered glass slides with pattern arrays of either pits (diameter ranging from 5-15 μm and depth from 2.5-7.5 μm) or grooves (width ranging from 5-15 μm and depth of 5 μm). Unprocessed slides served as controls. Cells were cultured and seeded onto slide chambers at 5,000 cells/cm². Surface characteristics and cell morphology were assessed using atomic force microscopy, and adhesion was observed using fluorescence microscopy. Cell population and viability were measured using an automated hemocytometer. Osteoinductive proteins DLX5, RUNX2, and SP7 were measured by Western Blot. Grooved and pitted patterns increased surface area by 20-70% and 10-45%, respectively. Feature size had the greatest influence on cell behavior, with 10 μm pits yielding ASC populations that were 83.1±8.9% higher than controls. Qualitatively, fluorescent indicators of focal adhesions suggested increased cell attachment on pits compared to grooved patterns. Compared to controls, both patterns lengthened the life cycle of DLX5 2-fold, which increased production of SP7 in ASCs and OBs by 79.0±4.2% and 68.9±12.2%, respectively. These results support future research on modifying implant surfaces to improve ossification.

Samples of bone were first machined with 1.5 cm×1.5 cm squared cross sections having a thickness of 3.0 mm. Laser technology described hereinabove was used to inscribe patterns including channels (i.e., grooves) having a width of 100 μm, a height of either 50 nm, 150 nm, or 300 nm, and a center-to-center pitch of 200 μm or pits having a diameter of 3 μm and a depth of 5 μm or 10 μm. Some samples including channels included channels that were straight up and down with respect to a side of the bone sample, while other samples included channels that were angled at approximately 45 degrees with respect to a side of the bone sample.

Once the sample machining and laser pattern applications were complete, the samples were exposed to a 2-step sterilization process. The samples were first submerged in 70% alcohol for a period of 2-4 hours. Samples were then removed from the alcohol, permitted to dry and placed under ultraviolet light for a period of 24 hours. The samples were then considered ready for cell seeding.

In this example, primary human osteoblasts were the only cell line studied. Cells were seeded onto five of the bone samples at approximately 5,000 cells per sample. Cells from the same batch were also seeded into the wells of a 6-well tissue culture treated dish that would be used as a control throughout the experiment. Five time points (24 hrs., 48 hrs., 72 hrs., 96 hrs. and 7 days) were studied in this initial example, in which one bone sample and one control well were analyzed for each time point. Through the course of the 7 day experiment, cells were maintained (regardless of scaffold) by replenishing the media, until their desired time point was reached, every 48 hours with pre-warmed media consisting of: 500 mL of DMEM/Ham's F-12 (Sigma-Aldrich CAT D6434), 10% Fetal Calf Serum (FCS) (Sigma-Aldrich CAT C8056), 1% Penicillin-Streptomycin-Glutamine (Thermo-Scientific/Hyclone Labs CAT SV30082.01) and 0.2% Amphotericin B (Thermo-Scientific/Hyclone Labs CAT SB30078.01).

Once growth and culture were complete, all samples were properly prepped for their prospective characterization methods.

All cell counting and viability assessments throughout the cell seeding, culture and maintenance were performed via standard optical microscopy. A fluorescence microscope is an optical microscope that uses fluorescence and phosphorescence instead of, or in addition to, reflection and absorption to study properties of organic or inorganic substances. In order for a sample to be suitable for fluorescence microscopy it must be fluorescent. There are several methods of creating a fluorescent sample; the main techniques involve the expression of a naturally fluorescent protein are labeling with fluorescent stains.

For this example, there were three initial intercellular targets of interest: actin filaments, focal adhesions and nuclei. Actin filaments are the central part of the actin cytoskeleton, a dynamic structure that rapidly changes shape and organization in response to stimuli and cell cycle progression. Focal adhesions are membrane-associated complexes that serve as nucleation sites for actin filaments and as cross-linkers between the cell exterior, plasma membrane and actin cytoskeleton. The function of focal adhesions is structural, linking the extracellular matrix (ECM) on the outside of the cell, to the actin cytoskeleton on the inside. They are also sites of signal transduction, initiating signaling pathways in response to adhesion. Focal adhesions consist of integrin-type receptors that are attached to the extracellular matrix and are intracellularly associated with protein complexes containing vinculin (universal focal adhesion marker), talin, α-actinin, paxillin, tensin, zyxin and focal adhesion kinase (FAK). Lastly, cell nuclei are the control centers of cells, containing the cell's chromosomal DNA. They regulate all cellular movements, changes and reactions to the cell's environment. Millipore's Actin Cytoskeleton and Focal Adhesion Staining Kit (Catalog Number FAK100) provided all necessary reagents to stain the actin filaments, vinculin and nuclei of the samples and prepare them for visualization via fluorescence microscopy.

The targets of interest to be assessed via the automated western blotting technique described hereinabove included Bone Morphogenetic Proteins (BMPs) and Integrins. BMPs are a group of growth factors that were originally discovered by their ability to induce formation of bone and cartilage. Of particular interest were BMP1, BMP2, BMP7 and BMP8a. Each of these is unique in its chromosomal location and structure, but each BMP has distinctive functions that make its presence crucial in bone regeneration. BMP1 acts as a pro-collagen and is involved in cartilage and fibrous tissue formation. BMP2 induces bone and cartilage formation and plays a key role in osteoblast differentiation. BMP7 also plays a key role in osteoblast differentiation and induces the production of SMAD1. SMAD1 is crucial in bone development because its key function is to transmit BMP signals in and out of cells. Lastly BMP8 not only induces bone and cartilage formation, but also is one of the primary factors responsible for ossification (the process of laying down new bone).

Integrins β1, β3 and β4 were also of particular interest. Integrin β1 is considered a fibronectin receptor; Fibronectin is one of the main glycoproteins on the surface of cells that acts as the “glue”, holding together the various components of the extracellular matrix. Integrin β3 also participates in cell-surface-mediated signaling, and integrin β4 may be important in collagen communication and interaction.

The initial characterization scheme consisted of the various microscopies described hereinabove in addition to some other fundamental cell studying techniques. Because of the unknown reactions associated with using a bone based substrate, the original characterization scheme was aborted. Bone's natural composition causes a strong degree of autofluorescence which makes all of the fundamental techniques and stains incorporated in the original characterization scheme deficient. It was therefore determined to add objectives to the project to determine the degree of autofluorescence, as well as how to accurately characterize cells adhered to bone based substrates that account for the autofluorescence issue.

Prior to any additional experimentation involving bone, it was decided to explore the same cell type grown on chambered slides with lasered patterns on the surface. This would help isolate the patterning as a treatment and determine its singular effect on the growth and adhesion of the cells. The results of this methodology will then be used in the next set of experiments, where bone will be the sole substrate for examination. Using traditional culture treated dishes will allow some of the standard characterization techniques such as fluorescent microscopy to once again be used.

As previously mentioned, the unexpected autofluorescence of bone forced an addition to the scope of this experiment. A pilot experiment involving cells grown on lasered bone substrates revealed the autofluorescence of bone. This led to the exploration of the degree of autofluorescence, as well as how the autofluorescence changes with respect to bone type, amount of bone present and wavelength to which the bone is exposed.

In the initial analysis of the autofluorescence of bone, the objective was to study the optical density of both cortical (CO) and cancellous (CA) bone, weighing equal amounts, under various wavelengths. A total of 36 samples (18 CO and 18 CA) each weighing approximately 200 mg were placed in separate wells of a 48-well non-culture treated plate according to the map shown in FIG. 2. The negative controls were simply left blank, which is indicated by NA in FIG. 2.

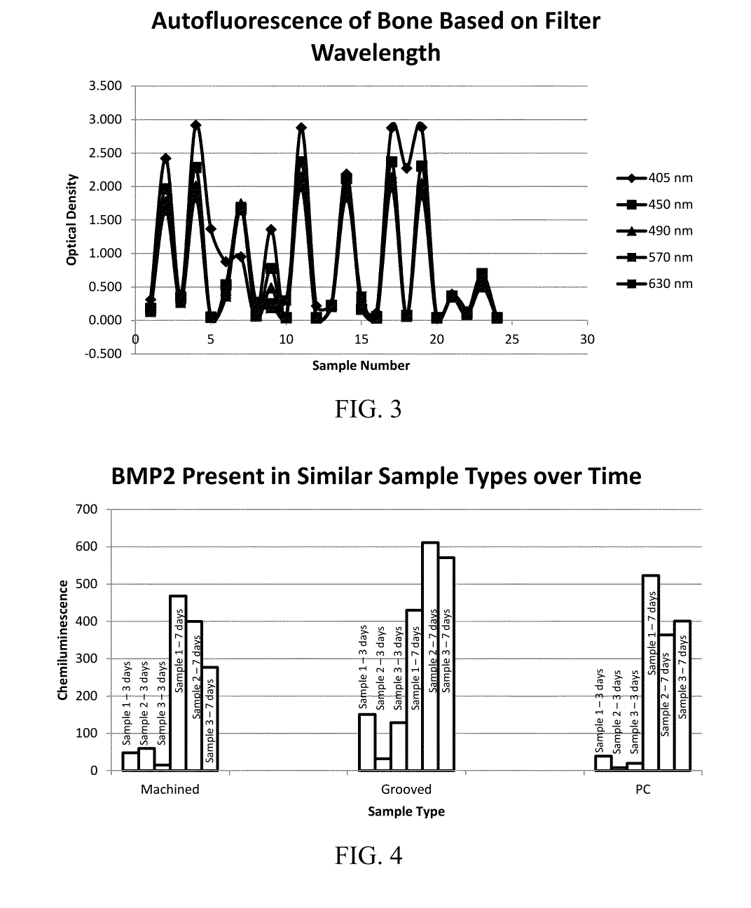

The plate was exposed to the following filters in a microplate reader: 405 nm, 450 nm, 490 nm, 570 nm and 630 nm, with the results illustrated in FIG. 3.

FIG. 3 combines all samples to compare optical densities as a function of filter wavelength. Only 24 out of the 36 samples produced an optical density in the linear range of 0.0-3.0. Anything beyond that range cannot be considered an accurate measurement of fluorescence. From FIG. 3, it was determined that the higher the filter wavelength, the lower the autofluorescence. The drastic differences between samples, illustrates the difference in autofluorescence between cortical and cancellous bone. The cortical samples all had much higher optical densities than those of the cancellous bone. This comparison of cortical versus cancellous is realistic and expected since cortical bone is much more dense than cancellous; the more bone matter present, the higher the amount of autofluorescence.

The next step was to determine what role the weight of the bone played in the overall autofluorescence. A titration of bone weights for both cortical and cancellous bone was designed to determine what weight of the bone (either cortical or cancellous) produced an optical density of 1.0. It could then be established that by used that weight or less, there would still be enough of the linear optical density spectrum remaining to calculate important properties using any form of colorimetric assay. From this analysis, it was determined that when exposed to a filter of any wavelength, cortical bone weight must be ≦10 mg, while the cancellous bone weight must be ≦15 mg in order to measure an optical quality using the microplate reader. It was also noted that bone at very small weights, was difficult to place in the wells since no liquid medium was present. This generated considerable electrostatic forces.

Utilizing the automated western blotting technique described hereinabove, the amounts of BMP1, BMP2, BMP7 and BMP8a were measured in the bone samples, along with Integrins β1, β3 and β4. BMP1, Integrin β1 and Integrin β4 had evident signals during the analysis across all sample types, but when compared to the positive control, no significant changes within the samples were seen over time. Although Integrin β3 is a key component of cellular adhesion, it is predominantly seen in thrombocytes; therefore, evidence of its existence in the studied samples was inconclusive.

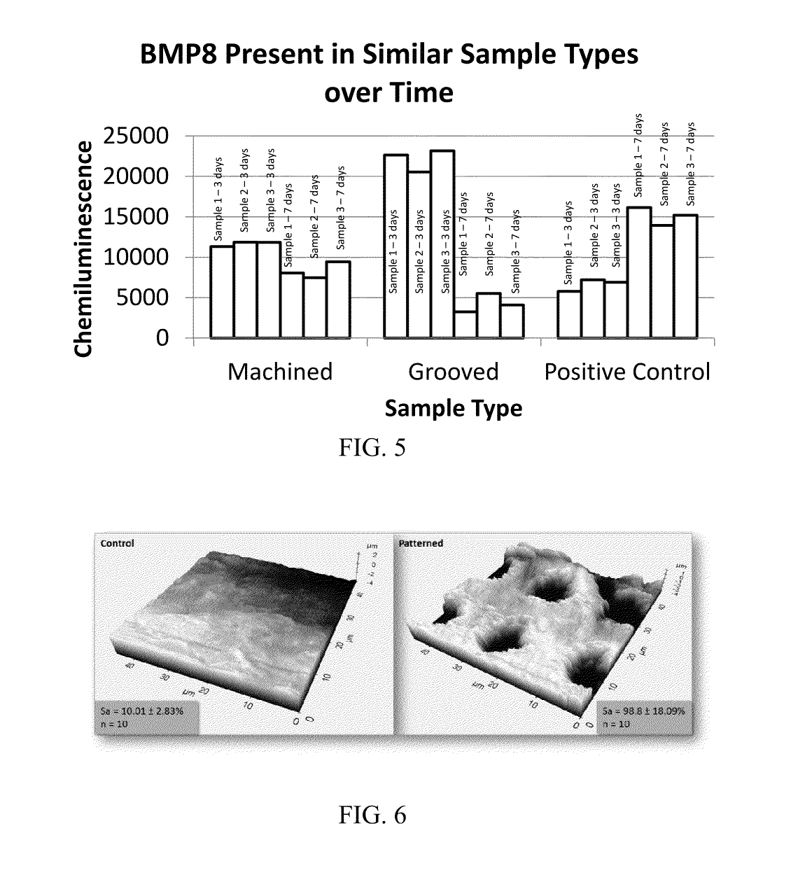

The most promising results were demonstrated by the presence of BMP2 and BMP8a. FIGS. 4 and 5 show how the prospective amounts of BMP2 and BMP8a changed over time within the same sample type. It was demonstrated that the patterning of the samples affected/promoted the production of these cellular targets.

The analyses performed with the microplate reader demonstrated the autofluorescence effect was on the bone samples. It also quantified the degree of autofluorescence to determine what characterization techniques could be applied in the future endeavors associated with this project. By titrating the amounts of bone present, it was possible to identify an acceptable sample size for which tests could be conducted utilizing fluorescence. Because these samples sizes were so small (cortical ≦10 mg and cancellous ≦15 mg), the utilization of fluorescent techniques for characterization is not dependably feasible.

The results demonstrated by the presence of BMP2 and BMP8a were very encouraging. After three days of culture, the cells that had adhered to machined and the grooved samples had significantly higher amounts of BMP2 present than just the cells that were grown on standard polystyrene culture treated dishes. After approximately seven days in culture, the amounts of BMP2 displayed in all sample types show a significant increase. When studying the amounts of BMP2 across the sample types at seven days, the quantities are more comparable to each other, however the grooved or patterned samples demonstrate increased amounts of BMP2 when compared to the other sample types. This is the desired result, since inscribed patterns on the bone surfaces increase the bone's surface roughness, and should therefore promote higher cellular adhesion. What remains interesting about the presence of BMP2 is the how the amount of BMP2 changes within a sample over time. As time progressed from 3-7 days, the amount of BMP2 present in the grooved samples increased by at least a factor of 3. In the machined samples, the amount of BMP2 increased by at least a factor of 5. Even though the amount of BMP2 increased by a factor of 10 within the control samples, the important thing to note is that the end result demonstrated that both the machined and grooved samples end up with higher amounts of BMP2. This indicates that more cellular activity and therefore more adhesion is taking place within those samples. These results are favorable for proving that modifying the bone's surface improves cellular adhesion and promotes successful integration between the cells and the bone based scaffold.

Similar results were seen when measuring the amounts of BMP8 among the samples. After merely 3 days, the amount of BMP8 in the machined samples was twice the amount present in the controls, and the amount present in the grooved samples was nearly four times the amount present in the controls. These results indicated that both the machined and patterned bone surfaces provide an advantage in the promotion of cellular adhesion. Interestingly, after 7 days in vitro, the amounts of BMP8 present in the machined and grooved samples dropped drastically, but in the positive control samples, it increased significantly. This behavior indicates a couple of possibilities: (i) the presence of BMP8 has a climactic window in which its presence is maximized on bone based substrates, and that optimal existence of BMP8 lies somewhere in the vicinity of 72 hours, and (ii) the production of BMP8 by osteoblasts is supremely promoted by bone based substrates, but has a much shorter half-life than any BMP8 molecules produced by cells grown on standard substrate materials.

FIGS. 4 and 5 highlight how the amounts of BMP2 and BMP8 progress in the various sample types of time. BMP2 seemed to increase over time in all of the sample types, and the amount by which it increased appears to be significant in all of the samples. The grooved samples appeared to have the highest amounts of BMP2 after 7 days of culture, showing significant promise in the utilization of BMP2 as a means to promote cellular adhesion on patterned bone.

The behavior of BMP8 appeared to be much different than that of BMP2. Instead of continuing to increase in amount, it appeared to drastically decrease on the machined and grooved samples, but increase in the control samples. It is possible the optimal time of presence for BMP8 is different than that of BMP2, or it is also possible that the modified sample surfaces associated with the machined and grooved samples promote much quicker cellular adhesion and mitosis, but ultimately exhaust a cell's potential for producing BMP8.

Both BMP2 and BMP8 are fundamental elements of successful cellular adhesion.

This example was to illustrate the positive reinforcement of bone as a scaffold. The results provided a positive foundation for the development of such scaffold. Although it was not originally intended, the framework of this study helped identify a significant problem associated with utilizing standard characterization techniques to study cellular adhesion to bone. Because of bone's natural autofluorescence, additional characterization methods had to be researched and applied. Identifying and quantifying the autofluorescence of bone in the early stages of this study has helped provide a more clear pathway to resolve in all future efforts to characterize bone based substrates.

Despite the presence of certain BMPs and Integrins being inconclusive, some of the more crucial BMPs did have a strong presence and illustrated that by enhancing the surface of the bone based substrate, the presence of those particular BMPs (BMP2 and BMP8) were also enhanced. In addition to proving the existence of these crucial BMPs, their optimal timeline with respect to presence within the sample was also identified. Since cellular behavior can often be simulated by a logarithmic pattern, cell growth normally starts slow, drastically increases in rate then finally tapers off. Since the development and expression of various BMPs is directly dependent on cell growth and proliferation, the availability of these BMPs is directly affected by the behavior of the cell population. It can now be assumed that the presence/availability of certain BMPs can also be simulated via a logarithmic type pattern.

### Example 2

The objective of this example was to illustrate the positive reinforcement of bone as a scaffold. It was concluded that the 10 μm feature size combined with a pitted pattern performed best with respect to cellular attachment, proliferation and protein expression. Therefore, these feature parameters were selected for further experimentation. Additionally, it was decided that future experiments would focus solely on stem cells to examine how their differentiation capabilities and ability to mature and promote regeneration are influenced by such patterns.

Cells were cultured using the cell culture method described hereinabove. Tubes of cryopreserved adipose derived adult stem cells (ASCs) were removed from the cryotank and placed immediately in a 37° C. water bath for 90 seconds with gentle agitation to thaw. Once cells were completely thawed, the cryotubes were sprayed with 70% alcohol and placed in sterile hood. Contents of all tubes were combined with 5 mL of media warmed to 37° C. and placed in 15 mL conical tubes. The cell/media suspension was centrifuged at 300×g for 5 minutes at room temperature. Tubes were then removed from the centrifuge and sprayed with 70% alcohol and placed in the hood. The supernatant was aspirated from each tube, being careful not to disturb each cell pellet. An adequate amount of fresh media was placed in each tube and the cells were resuspended within the media. The cells were then counted using a disposable hemocytometer to get an estimate of cell density per milliliter of media. This aided in formulating the amount of cell-media suspension needed on each bone sample to maintain a consistent cell seeding density.

Cortical bone blocks (1.0 cm×1.0 cm with approximately 5.0 mm thickness) were created as described hereinabove. In particular, the major faces of each block were end-milled in a CNC machine to create relatively smooth and parallel surfaces. Half of the blocks were randomly selected for the patterned (treatment) group; these were laser micromachined to inscribe the pattern defined in FIG. 1. The pits measured 10.0±0.1 μm in diameter, with a depth equal to half the diameter and a pitch of 20 μm. After machining, all blocks were sterilized by soaking in 70% isopropyl alcohol for 4 hours followed by drying under ultraviolet light for 24 hours.

Adipose-derived stem cells were seeded at a density of 5,000 cells/sample. Eight time points (48 hrs., 72 hrs., 96 hrs., 7 days, 10 days, 14 days, 17 days and 21 days) were studied, in which 5 treated samples and 5 control samples were analyzed for each time point, yielding a total of 80 samples. Through the course of the 21 day experiment, cells were maintained (regardless of scaffold) by replenishing the media until the desired time point was reached. The media consisted of: 500 mL of DMEM/Ham's F-12, 10% Fetal Calf Serum (FCS) and 1% Penicillin-Streptomycin-Glutamine.

Once the appropriate time point was reached, cells were removed from all samples and prepped for characterization. Specifically, all old nutrient essential media was aspirated from the system (bone and cells) and rinsed once in phosphate buffered saline (PBS). The PBS was removed after rinsing and a solution including 0.25% trypsin and 0.05% EDTA was added to the system in an amount to completely cover the growth surface. The system was then covered and placed in an incubator at 37° C. for 6-7 minutes. The system was removed from the incubator and the dishes containing the bone, cells and trypsin/EDTA solution were gently nudged to break loose any cells still attached.

Next, an equal amount of complete cell medium (DMEM and Ham's F-12, 10% fetal bovine serum (FBS) and 1% penicillin-streptomycin) was added to neutralize the action of the trypsin/EDTA mixture. The bone pieces were turned over using sterile forceps so that the surface that was seeded with cells (and has the pattern for the treated group) was face down. The bone pieces were forcefully tapped 4-5 times using the blunt end of the forceps to release any cells still attached. The bone pieces were then removed and set aside. The solution, including the cells, was aspirated and in a 15 mL centrifuge tube and centrifuged at 2700 rpm at room temperature for 10 minutes. After centrifugation, the supernatant was removed with care to not disturb the cell pellet. The pellet was then resuspended in 1 mL of fresh media and stored at −4° C. until protein characterization. Bone blocks were returned to their original wells and kept refrigerated until surface characterization.

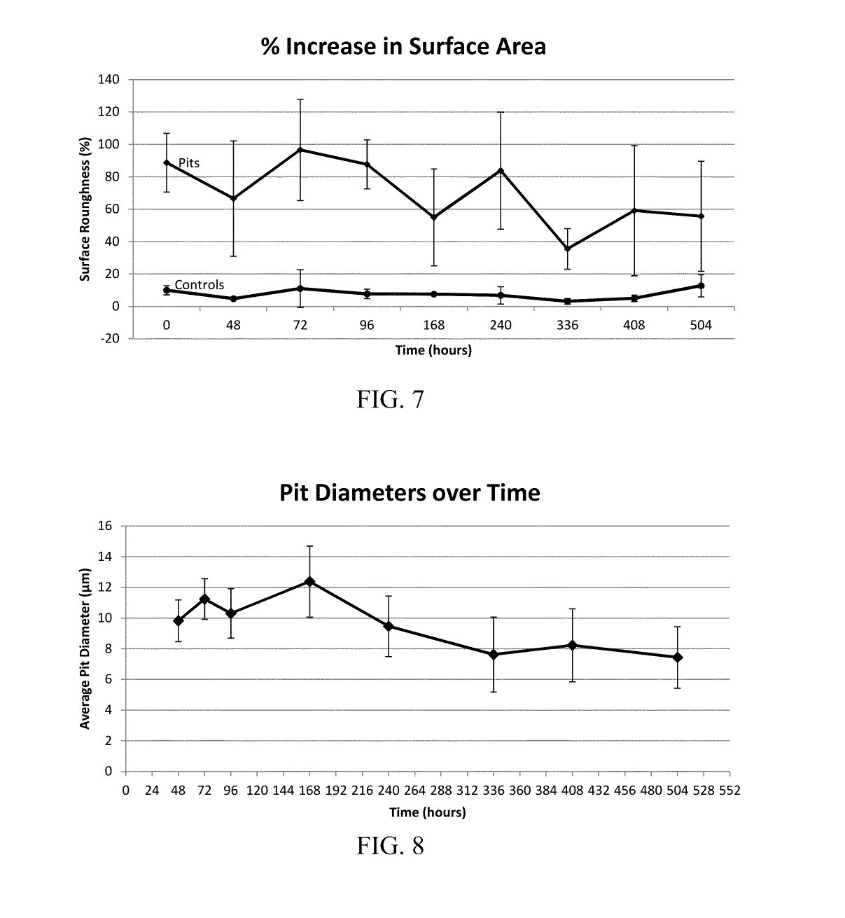

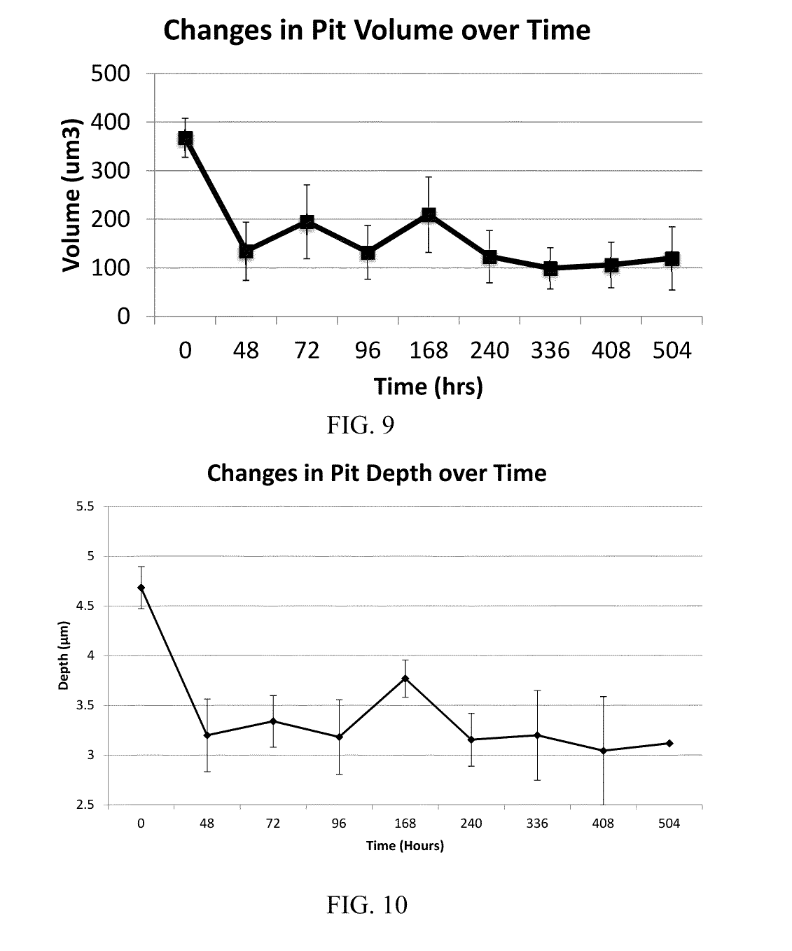

Each sample's surface post-cell seeding was characterized using atomic force microscopy. Specifically, each sample was scanned over five scan areas each sized 45 μm by 45 μm at a scan rate of 0.3 Hz. The surface roughness measurements and geometrical area and actual surface area were documented for all scans. Average surface area on patterned samples was increased by 88.7±18.12% compared to controls, as shown in FIG. 6. After a 72 hour exposure to ASCs, the average surface roughness on patterned samples was 96.6±31.3% greater than the calculated geometrical area as opposed to only 11.0±11.6% greater on control samples, as shown in FIG. 7. At 7 days, bone resorption on patterned samples was maximized at 737.4±408.9 μm², as evidenced by an overall increase in pit diameter of 12.4±2.3 μm², as shown in FIG. 8. After 21 days, patterned bone formation was 384.45±226.1 μm² and pit diameter had reduced to 7.4±2.0 μm². Pit volume and depth changes parallel those in surface roughness, with notable increases within the first 72 hours followed by leveling out at just over 100 μm³ and 3 μm, respectively, as shown in FIGS. 9 and 10.

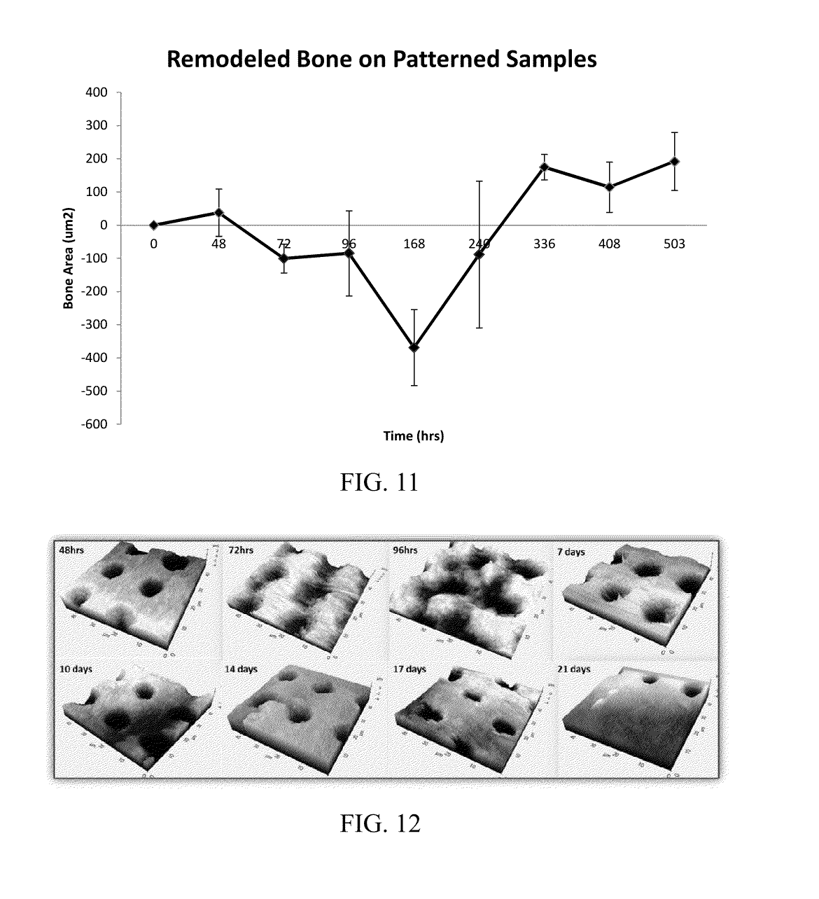

By monitoring the changes in the volumetric and surface roughness parameters, a plot of bone resorption and bone growth over time was generated demonstrating the course of the bone remodeling over the 21 day experimental period (FIG. 11). Maximum resorption occurs at 7 days (168 hours), followed by a remodeling/growth phase peaking at maximum bone surface area at 21 days (504 hours). FIG. 12 illustrates the progression of bone resorption to bone formation over the course of 21 days on patterned samples.

RUNX2 (Runt Related Transcription Factor 2) plays a crucial role in promoting osteoblastic differentiation by diverting cells from the myogenic pathway. SP7/Osterix is a zinc finger DNA binding protein located downstream of RUNX2 that promotes cell maturation in the osteoblastic lineage, specifically immature pre-osteoblastic cells. It is pivotal in bone cell maturation and matrix mineralization. Accordingly, RUNX2 and SP7 were measured using an automated Western blot system, which detects and quantifies protein content as a chemiluminescent signal. While no statistically significant differences were observed in RUNX2 production between patterned and control groups, there was a trend for it to be higher for patterned samples at and beyond the 10 day timepoint. SP7 production was higher at all time points on patterned samples, and statistically different from the controls at both 7 and 10 days (p=0.05 and p=0.0006 respectively).

Patterned samples were statistically higher in surface roughness throughout the entire experiment, giving cells more opportunity for adherence and involvement in bone turnover. The changes in both pit depth and pit volume coincided with the changes seen in surface roughness, and were maximized at the same time point in which maximum resorption was noted. This further endorses that the actual process of turnover is taking place.

The maximum bone resorption occurs at a time that congeals well with the maximum phosphorylated SMAD signaling of 72 hours, which was discovered on the glass slide study. As the SMADs carry the BMP messages within the cell, and the cells respond, the time that they respond, by adjusting to the environment, coincides with the time at which all of the remodeling hits a maximum. This can also be seen by the dip in SP7 production at the same time that max resorption occurs. That dip in SP7 production indicates the cells aren't as interested in maturing at that point, because they are more focused on producing the necessary proteins to assist in resorption. That behavior is followed by a steady increase in both measured proteins (RUNX2 and SP7) from that point until the experiment's completion.

From this example, it was concluded that the adipose stem cells respond optimistically to the patterned bone surface. The strong presence of RUNX2 demonstrates that the stem cells are differentiating down the osteoblastic lineage, to assist with bone turnover. The stronger presence of SP7 in the patterned versus the control samples aids the stem cell differentiation by helping to mature those cells toward osteoblasts and osteocytes.

AFM scans over the course of the 21 days showed a significant change in surface roughness. Resorption of the bone was confirmed by the increased surface roughness maxing out 7-10 days. This behavior is followed up with a gradual increase in bone area, suggesting that bone formation is actually taking place. This was confirmed with supporting AFM scans, such as those shown in FIG. 12.

### Example 3

Cells were cultured using the cell culture method described hereinabove. Tubes of cyropreserved ASCs, FBs, and BMSCs were thawed by gentle agitation at 37° C. for 90 seconds. Once thawed, cells were sprayed with 70% alcohol and placed in a sterile hood. The contents of each tube were combined with 5 ml of media that had been pre-warned at 37° C. and centrifuged at 2700 rpm for 5 minutes. Taking care not to disturb the cell pellet, the supernatant was discarded and fresh pre-warmed media was used to resuspend the cells prior to counting with a hemocytometer. After counting, an additional amount of fresh media was added to achieve a cell density of 5000 cells/20-30 μl. The appropriate microliter volume of cell suspension was applied to each bone block to achieve a seeding density of 5000 cells/bone block.

Cortical bone blocks (1.0 cm×1.0 cm with approximately 5.0 mm thickness) were created as described hereinabove. In particular, the major faces of each block were end-milled in a CNC machine to create relatively smooth and parallel surfaces. The blocks were then terminally sterilized. Half of the blocks were randomly selected for the patterned (treatment) group; these were laser micromachined to inscribe the pattern defined in FIG. 1. The pits measured 10.0±0.1 μm in diameter, with a depth equal to half the diameter and a pitch of 20 μm. After machining, all blocks were sterilized by soaking in 70% isopropyl alcohol for 4 hours followed by drying under ultraviolet light for 24 hours.

ASCs, FBs, and BMSCs were studied by seeding at a density of 5,000 cells/human cortical bone block. Four time points (72 hrs., 7 days, 10 days, and 21 days) were studied, in which 5 treated samples and 5 control samples were analyzed for each time point, yielding a total of 120 samples. Throughout the 21 day experiment, cells were maintained by replenishing media until the desired time point was reached. The media consisted of: 500 mL of DMEM/Ham's F-12, 10% Fetal Calf Serum (FCS) and 1% Penicillin-Streptomycin-Glutamine.

Once the appropriate time point was reached, cells were removed from all samples and prepped for characterization. Specifically, all old nutrient essential media was aspirated from the system (bone and cells) and rinsed once in phosphate buffered saline (PBS). The PBS was removed after rinsing and a solution including 0.25% trypsin and 0.05% EDTA was added to the system in an amount to completely cover the growth surface. The system was then covered and placed in an incubator at 37° C. for 6-7 minutes. The system was removed from the incubator and the dishes containing the bone, cells and trypsin/EDTA solution were gently nudged to break loose any cells still attached.

Next, an equal amount of complete cell medium (DMEM and Ham's F-12, 10% fetal bovine serum (FBS) and 1% penicillin-streptomycin) was added to neutralize the action of the trypsin/EDTA mixture. The bone pieces were turned over using sterile forceps so that the surface that was seeded with cells (and has the pattern for the treated group) was face down. The bone pieces were forcefully tapped 20 times using the blunt end of the forceps to release any cells still attached. The bone pieces were then removed and set aside. The solution, including the cells, was aspirated and in a 15 mL centrifuge tube and centrifuged at 2700 rpm at room temperature for 10 minutes. After centrifugation, the supernatant was removed with care to not disturb the cell pellet. The pellet was then resuspended in 1 ml of fresh media and stored at −4° C. until protein characterization. Bone blocks were returned to their original wells and kept refrigerated until surface characterization.

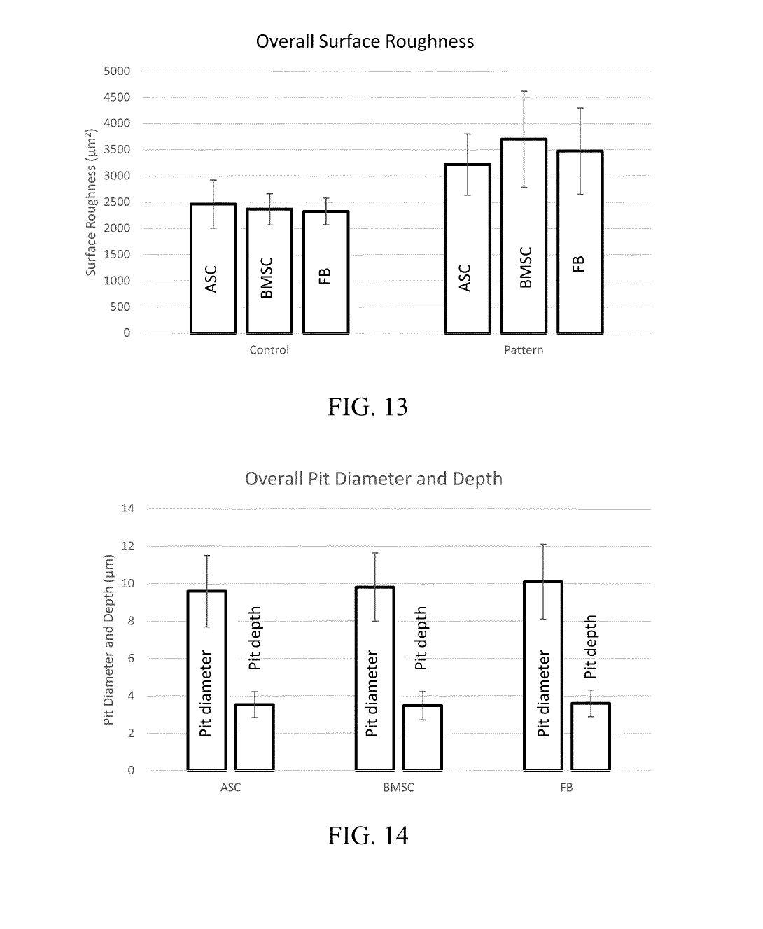

Each sample's surface post-cell seeding was characterized using atomic force microscopy. Specifically, each sample was scanned over five scan areas each sized 45 μm by 45 μm at a scan rate of 0.3 Hz. The surface roughness measurements and geometrical area and actual surface area were documented for all scans. Surface roughness on patterned samples was maximized at 3475.7844±691.183 μm² after 10 days of treatment with ASCs, 22% greater than control surface roughness. Patterned surface roughness was significantly greater (p<0.002) at all time points. Bone resorption was maximized in patterned samples after 72 hours exposure to BMSCs at a surface roughness of 4419.8289±977.5923 μm², 92% greater than control surface roughness. At 10 days, surface roughness has decreased to 25% greater than controls, with the 72 hour patterned surface roughness being significantly greater than the 10 day patterned surface roughness (p<0.001). Patterned surface roughness was significantly greater than controls at all time points (p<0.001). Bone resorption was maximized in patterned samples after 72 hours of exposure to FBs at a surface roughness of 3826.1447±991.109 μm², 53% greater than control surface roughness. Patterned surface roughness was significantly greater than controls at all time points (p<0.001). Patterned surface roughness is significantly greater in bone blocks treated with BMSCs (p<0.001) and FBs (p=0.044) compared to bone blocks treated with ASCs and control surface roughness was significantly greater in bone blocks treated with ASCs compared to FBs (p=0.047), as shown in FIG. 13.

Bone resorption was maximized after 72 hours of ASC exposure based upon a 10% increase in pit diameter. The 10.957±2.161 μm² diameter at 72 hours was significantly greater (p<0.001) than the pit diameter at all other time points. Patterned bone formation was demonstrated by an 11% reduction in pit diameter to 8.906±1.589 μm after 21 days. Bone resorption was maximized after 72 hours of BMSC exposure, supported by a 5% increase in pit diameter. The 10.511±1.818 μm² diameter at 72 hours was significantly greater (p<0.001) than the pit diameter at 10 and 21 days. Changes in pit depth and diameter parallel the changes in surface roughness. Bone formation is demonstrated by an 8% reduction in pit diameter and 33% reduction in pit depth after 21 days of treatment. Bone resorption was maximized at 72 hours in FB exposed patterned bone blocks, supported by a 12% increase in pit diameter. The 11.233±2.082 μm² diameter at 72 hours is significantly greater (p<0.001) than the pit diameter at all other time points. Changes in pit depth and diameter parallel the changes in surface roughness. Bone formation was demonstrated by 4 and 33% decreases in pit diameter and depth, respectively, at 21 days. Overall, pit diameters were significantly larger in BMSCs (p=0.014) than ASCs and FB pit diameters were significantly larger than both ASC and BMSC pit diameters (p≦0.026). Pit depth was significantly larger in FBs compared to BMSCs (p=0.003), as shown in FIG. 14.

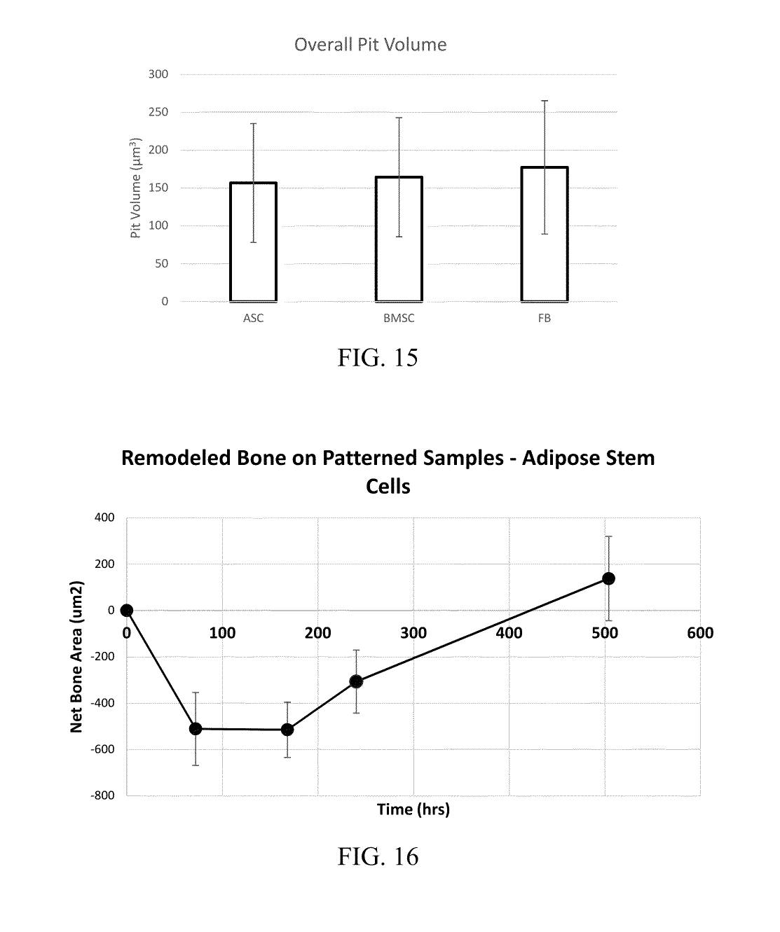

Pit volume changes in BMSC exposed patterned blocks are consistent with the changes in pit diameter seen above, leveling out at 10 days at approximately 130 μm³. Pit volume changes in BMSC exposed patterned blocks are consistent with the changes in pit diameter seen above and changes in surface roughness seen above, leveling out at 10 days at approximately 150 μm³. Pit volume changes in FB treated patterned bone are consistent with the changes in pit diameter seen above and changes in surface roughness seen above, leveling out at 7 days at approximately 160 μm³. Overall, pit volume was significantly greater in FB exposed patterned bone than both BMSC and ASC treated bone (p≦0.023), as shown in FIG. 15.

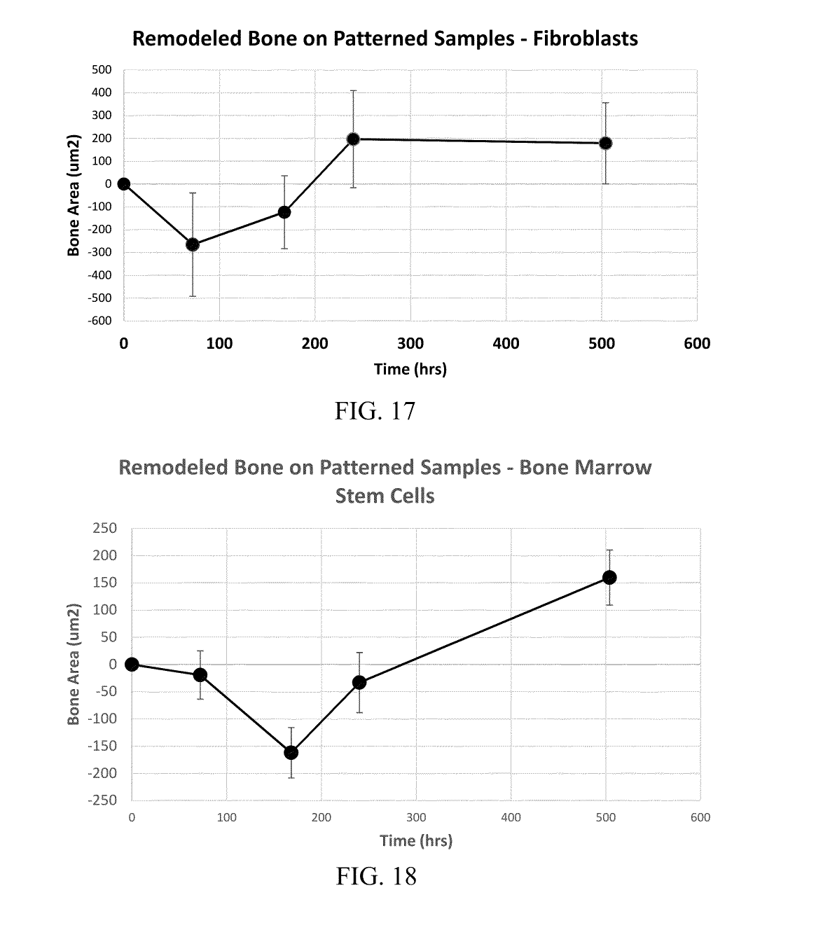

The changes in pit depth, diameter and volume over time, along with the original pit parameters and surface roughness were used to gage the net change in bone volume over time for all three cell types. The ASCs show a sharp and intentional bone resorption pattern within the first 72 hours. The resorption time continued for the next few days, followed by a slow but deliberate increase in bone until remodeling (i.e., bone growth) was actually taking place by approximately 18 days, as shown in FIG. 16. The fibroblasts showed a similar behavioral pattern with a short term period of resorption followed by a bit more drastic increase in bone growth. The fibroblasts only resorbed for a short period of time, and the amount of resorption topped out at just about half of the amount of resorption achieved by the ASCs, as shown in FIG. 17. This was followed by maximized bone growth at 10 days at which time the bone reached a state of homeostasis, where the amount of bone remained constant for the next several days. The BMSCs performed similarly to the ASCs, but with a bit of a delayed maximum resorption period and a slightly more drastic change from resorption to bone growth, as shown in FIG. 18. The BMSCs show amounts of maximum resorption similar to the FBs, but with more potential for bone growth, since their bone area was still trending upward at the conclusion of the study.

Each cell type was analyzed for the amounts of each protein it produced at all 4 time points. The ASCs exhibited significant amounts of SP7 at 72 hours (p=0.0234). At 7 days, the ASC controls were significantly higher in RUNX2 production (p=0.0097) while the patterned ASCs again produced a significantly higher amount of SP7 (p=0.004789). The higher SP7 production demonstrated more osteoblast maturation in the patterned samples. After 10 days, several proteins produced by the ASCs on the patterned bone were significantly higher than their equivalent controls (SP7, macrophage colony stimulating factor (M-CSF), tartrate resistant acid phosphatase (TRAP), cathepsin K (CTSK) and E3 ubiquitin ligase tumor necrosis factor receptor associated factor 6 (TRAF6)). The increase in these proteins was indicative of more osteoclastic behavior, including macrophage formation, osteoclast formation and activation and bone resorption. At 21 days, the ASCs on patterned bone were still producing significantly more amounts of some osteoblastic (SP7, p=0.0313) and osteoclastic (M-CSF, p=0.0043 and TRAP, p=0.0144) proteins.

The FBs surprisingly demonstrated some activity as well across most time points. At 72 hours, the control samples exhibited higher amounts of TRAP, receptor activator of nuclear factor (RANK), CTSK and receptor activator of nuclear factor kappa-B ligand (RANKL). These proteins speak more about osteoblastic signaling and migration. At 7 days, however, only 3 of the 8 proteins measured were being produced by fibroblasts (RUNX2, SP7 and M-CSF). The patterned FBs were demonstrating a significantly higher amount of M-CSF while the controls did not produce any. The unique behavior of the fibroblasts continues after 7 days, when all proteins produced are significantly higher in the patterned samples versus the controls. Lastly, at 21 days, the samples of the two treatments behaved similarly with nothing truly significantly different between them.

The BMSCs presented behavior similar to the ASCs, yet with more significant protein production at earlier time point. SP7 and M-CSF remained significantly higher in the patterned samples over the controls through the first 7 days of the study. Also at 7 days, RUNX2 and CTSK were significantly higher in the patterned samples (p=0.0449 and p=0.0017 respectively). The controls produced significantly more TRAP and TRAF6 after 7 days (p=0.00215 and p=0.0203). Behavior between the two treatments at 21 days was similar with nothing being significantly different.

In this example, patterned samples were significantly greater than controls in surface roughness throughout the course of the experiment for all cell types, indicating that the increased surface roughness improved cell adherence and bone remodeling activity. Measures of pit volume, depth, and diameter coincided well with surface roughness changes for BMSCs and FBs but not for ASCs, endorsing the greater response seen with cells that are more likely to be first responders to an implant site.

Just as in the previous example, the ASCs resorbed bone quickly before rebuilding. In this example, they resorbed bone longer than the other two cell types, despite achieving similar bone growth as BMSCs, as shown in FIGS. 16 and 18. This could indicate that the resorption rates with ASCs are slower than that of BMSCs. FBs also exhibited resorption capabilities, but they were maximized at about half the capability of the ASCs, as shown in FIG. 17. This behavior was somewhat expected since FBs are more of a primary cell line. Ultimately, the patterned surface seems to have a positive impact on all three cell lines, with healing/turnover occurring most quickly in the BMSCs.

With the ASCs, SP7 is significantly higher in the patterned samples over the controls throughout the entire study. This is indicative of heightened osteoblastic maturation. This is crucial for bone turnover since mature osteoblasts are responsible for providing the proteins (RANKL) necessary to generate activated osteoclasts. At latter time points, the ASCs have significantly more M-CSF and TRAP on the patterned samples, which represents intensified macrophage formation and osteoblast migration. Both of these functions are crucial in activating osteoclasts to resorb bone. Increased TRAF6 indicates increased signaling between macrophages and osteoblasts, which is also critical for forming osteoclasts and bone resorption.

The FBs demonstrates a wide variety of protein expression, some of which was unexpected. The FBs immediately produce both osteoblastic and osteoclastic markers (TRAP, RANK, CTSK and RANKL) all of which are significantly higher on the controls after 72 hours. These proteins are suggestive of osteoblast migration, osteoblast/osteoclast communication and bone resorption. This early behavior on controls is probably due to a couple factors including: (i) the cells present do not need to be differentiated or matured since they are already a more primary cell line than any stem cell and (ii) the control surface only has a natural roughness, so what little remodeling needs to be completed can start immediately since stem cell differentiation, macrophage development and osteoclast formation are not required. Essentially turnover initialization happens quicker with the FBs, but not as much resorption takes place (FIG. 17). By 10 days, the FBs on the patterned surfaces are producing all 8 proteins at levels significantly higher than the controls. Since the control surfaces had less opportunity for resorption and new bone formation, this makes sense in that the patterned surfaces have more potential for remodeling and will take longer to complete their task. Lastly, it was encouraging to see such a wide range of proteins being produced by a more devoted cell line. Perhaps the patterned bone helps induce or increase the pluripotent potential of FBs in order to behave more like osteoblasts and undifferentiated cells.

The performance of the BMSCs was most telling since they are likely to be the first responders in a bone remodeling condition. Initially the RUNX2 produced by the BMSCs was higher on the control samples, but by 7 days, this trend was eliminated and both SP7 and M-CSF were significantly higher in the patterned samples. These two proteins are again crucial for cell maturation and macrophage formation, the two functions that precede bone resorption. At 72 hours, maturation (SP7) and migration (TRAP) are significant in the patterned samples demonstrating cells moving to the resorption site and preparing to remodel. By 7 days, cells continue to mature (SP7) but macrophage formation (M-CSF) begins. At 10 days, SP7, M-CSF and CTSK are all significantly higher in the patterned samples. The existence of CTSK is telling of bone resorption. Lastly at 21 days, almost all markers were higher on the patterned versus the control samples indicative of the remodeling process coming full circle. Although the BMSCs do not seem to take as much time to remodel, they reach a state of net growth, or new bone formation just 12 days into the study (FIG. 18, when the plot crosses the x-axis), while the ASCs achieve just as much growth, but do not start forming new bone until approximately 18 days into the study (FIG. 16, when the plot crosses the x-axis). This demonstrates the pattern's ability to enhance the capabilities of BMSCs to achieve faster bone turnover.

### Example 4

The rabbit was considered an appropriate animal model for evaluating materials by the current ISO testing standards. The lateral condyle is of sufficient size to accommodate a single cancellous bone implant site, approximately 4 mm in diameter and would also mimic the site of clinical use. A maximum of two articles (one per implant site) can be implanted in each rabbit.

Due to the length of the study and the extent of the surgical procedure, nine rabbits were used to ensure enough sites were available at each termination interval for evaluation. There are no available validated in vitro assays or computer simulated models that can mimic the complexity of post-surgical tissue reaction.

The surgical site was draped, and using sterile technique the lateral aspect of the distal end of the femur over the lateral condyle was exposed through a routine surgical approach. An initial pilot hole (2 mm diameter) was created using a drill in the lateral aspect of the femoral condyle. Using a power drill with an approximate 4 mm drill bit, the hole was enlarged. Saline irrigation was used to remove loose bone fragments.

The defect had an approximate depth of 10-11 mm. One test article (control or patterned) was implanted in the right femoral condyle of each animal. The article was placed in the bone defect to fill the void. Test articles were allograft bone in the form of a pin. Control articles had no patterning while patterned test articles had pits having diameters of 10 μm. The muscle and/or fascia were closed with 4-0 absorbable suture and the skin was closed with a combination of surgical staples and absorbable suture. The animal was repositioned, the surgical site prepared, and the left femoral condyle of the rabbit were similarly operated and implanted with the opposite test article. The day of implantation was designated as day 0.

Post-surgery, each animal was moved to a recovery area, placed on a heat source, and monitored for recovery from the anesthetic. Once sternal recumbency was achieved, each animal was returned to its cage. Additional once daily doses of the NSAID ketoprofen (1.0 mg/kg) were given at the discretion of the veterinarian (3 days post-operatively). Each animal received another injection of the analgesic buprenorphine (0.05 mg/kg) at approximately 6 hours after the first injection. On days 1-3, another dose of enrofioxacin was administered at 10 mg/kg.

In the first two-post-operative weeks (±3 days), manipulation of the animals outside of the cage was kept to a minimum to allow for optimal healing of the implant sites. This included no cage changing and no body weights. The animals were only removed from the cage if necessary for conducting observations, removing/replacing wound clips, and if medical treatment was needed. Animals were observed daily for general health, and body weights were recorded prior to implantation, at week 2, every four weeks thereafter, and prior to termination.

At 4 weeks, four animals were arbitrarily selected, weighed, and euthanized with an intravenous injection of a sodium pentobarbital based euthanasia solution. The implant sites and adjacent muscle tissue were examined macroscopically and the observations were recorded. Any adverse observations at the implant sites were described. Each femur was dissected free and removed. For each femur, an approximate 5 mm long medial section was removed from the implant site followed by the remaining lateral section, which was placed in 10% neutral buffered formalin (NBF). At 8 weeks, the remaining animals were similarly euthanized, examined, and processed. The remaining tissue from the lateral section of the implant site was immediately frozen for further analyses.

The unfixed tissue from both 4 and 8 week animals was thawed and the implanted pin was separated from the surrounding condylar bone. Protein was extracted from these tissues. Specifically, all samples were sterilized by dipping in 70% ethanol for 15 seconds. Each sample was washed with 1×-PBS five times for three minutes each time. Samples were frozen (<−80° C.) and crushed with a bio-pulverizer prior to weighing on an analytical balance. Samples were submerged in collagenase solution and vortexed prior to a 20 minute incubation at 37° C. The collagenase solution had a concentration of 1 mg/mL type 1 collagenase in isolation buffer. Isolation buffer includes 25 mM HEPES buffer (pH 7.4), 70 mM NaCl, 10 mM NaHCO₃, 60 mM sorbitol, 30 mM KCl, 3 mM K₂HPO₄, 1 mM CaCl₂, 1 mg/mL BSA, and 5 mg/mL glucose. Samples were vortexed again prior to centrifugation at 200×g for 5 minutes. The supernatant was collected and retained for analysis. Submersion, vortexing, centrifugation, and collection of the supernatant was repeated three more times, and the supernatant fractions were combined each time.

Each pin's pre-implantation surface was characterized using atomic force microscopy. Each pin was scanned eight times (two times on each face). Scan areas were each sized 20 μm by 20 μm and scanned at a scan rate of 0.3 Hz. The surface roughness measurements were documented for all scans.

After adequate fixation, the lateral sections were decalcified, then processed for paraffin embedding, and sectioned. Slides were prepared from two levels within each block (one block per femur) and from each level three slides were prepared. One slide was stained with Hematoxylin and eosin (H&E), one slide was stained with Masson's Trichrome, and one slide was prepared unstained for possible future analysis.

A pathologist conducted the microscopic evaluation of the implant sites. The implant sites were evaluated for cellular reactions (including inflammation) to the test articles. Cellular changes i.e local tissue response were graded (0-4) based on the scoring scheme in ISO 10993, Part 6, Annex E, and new bone formation was also graded (0-4) based on a custom scoring scheme.

A different pathologist also evaluated the H&E and Trichrome stained slides to obtain another independent analysis. Sections of bone were analyzed for the integration of a bone implant in the femoral condyle.

All H&E (n=36) and Masson's Trichrome (n=36) stained slides were imaged using a high-resolution digital microscope. For each slide, the implant and immediate surrounding area (host interface) was imaged at four magnifications: 10×, 30×, 50× and 200×. DNA content was quantified from the H&E images, while the amounts of cancellous and cortical bone as well as non-mineralized cartilage were quantified from the Tri-chrome images.

DLX5, RUNX2, SP7, BMP8a, BMP2, and BMP7 were quantified using an automated western blot system, which detects and quantifies protein content as a chemiluminescent signal. The automated western blot system automates the Western blotting process, including sample loading, size-based protein separation, immunoprobing, washing, detection, and data analysis for up to 12 samples in the kits simultaneously. Total protein content was measured using a microplate reader to detect and quantify protein content from a photometric analysis.

Surface roughness and protein data were analyzed using a variety of statistical and diagnostic techniques. Student's t-test and the Pearson product moment correlation were used to compare the statistical significance between control and patterned samples across all time points. Raw chemiluminescent signal was adjusted by a correction factor generated from sample weights to correct for varying amounts of protein content.

All animals tolerated the procedure well with no adverse effects observed during the procedure. All animals had reduced fecal output for at least one day post implantation, suggesting decreased food consumption. This observation was attributed to the surgical procedures and the anesthetic. The animals were provided supplements as needed. All other clinical observations were considered minor and incidental.

Fifty-six percent of the animals lost body weight during the first two weeks of the experiment. Given the surgical procedures, this was not an unanticipated observation. With the exception of one animal, all animals gained body weight in comparison to their pre-treatment body weight and termination body weight. All animals maintained a healthy body weight throughout the experiment.

A lesion was found along the left lateral aspect of the mid-patellar groove of one animal. The area was roughened with an irregular surface and was along the same linear tract as the surgical defect site. Additionally, the contour of patella ridge was abnormal. The findings correlated to the clinical observations (medial patellar luxation). Otherwise, at 4 and 8 weeks, all adjacent muscle tissue (surrounding the implant sites) and all implant sites were considered macroscopically normal.

Microscopically, the response to both articles was equivalent and the treated allograft bone pins were considered a nonirritant as compared to untreated allograft bone pins at 4 and 8 weeks. At 4 and 8 weeks, the average bone growth score for both control and treated implants was 1.0 (minimal).

The additional microscopic review noted that all implants in the area where there is contact with native bone have a thin zone of blue staining on the Tri-chrome section. This compares to the prominent red staining throughout the implant. There was a definitive zone of bone present around all implants. Few to occasional osteoblasts were observed on the recipient surface adjacent to the implant, with no apparent differences between treated and control implants. Some cellular infiltration into the Harversian canals was noticed, and mild to moderate increased cellularity in the bone marrow adjacent to the implant was observed in most specimens.

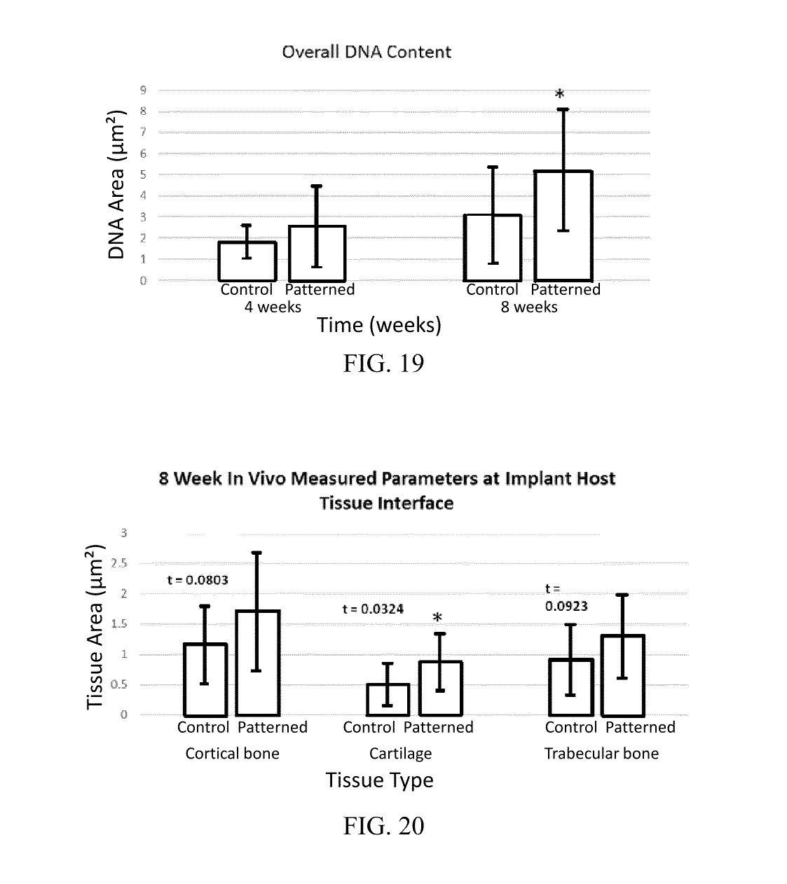

Utilizing IMAGEJ's “BoneJ” plugin, DNA content was quantified at both 4 and 8 weeks in all samples. Patterned samples had higher amounts of DNA at both time points, but the difference was only significant at 8 weeks, as shown in FIG. 19. By thresholding the histology images, the DNA content could be isolated and quantified. Across time, similar results were seen in overall DNA content, with the patterned pins exhibiting significantly more DNA than the control pins.

At the implant host tissue interface, amounts of cortical bone formation and trabecular bone formation were higher in the patterned pins, with cartilage formation being significantly higher in the patterned pins, as shown in FIG. 20. Cartilage formation at the interface is indicative of novel bone formation prior to mineralization and is therefore key to implant integration. In addition to the interface cartilage formation, the amount of cortical bone growth after 8 weeks was significantly higher in the patterned versus control pins. When the amounts of cortical bone, trabecular bone and cartilage were compared from 4 to 8 weeks, the “net” change in each for both the controls and the treated pins were examined. In all three tissue types, the patterned pins exhibited significantly higher amounts of tissue formation. Despite the overall histological observations demonstrating that the treatment and control pins behaved similarly, this quantitative image analysis identified increased amounts of DNA within the patterned pins as well as key increases in the formation of bone and cartilage at the pin/tissue interface and implant site in general.

Protein content in rabbit and pin tissue at 4 and 8 weeks post-implantation was measured. Many of the proteins tested showed significantly greater amounts for the 8 week time point than the 4 week time point (p≦0.036). At four weeks post-implantation, RUNX2 content in the condylar tissue was significantly greater in controls compared to animals with treated pins (p=0.000384). BMPs were significantly greater in treated tissues than in control tissues (p≦0.0427). Protein content in the pin is greater than that in the rabbit at both time points and for both groups (p<0.05). Pre-implantation surface roughness in patterned pins was positively correlated with 4 week rabbit tissue RUNX2 (0.965; p=0.0345) and negatively correlated with 8 week pin RUNX2 (−0.958; p=0.042). In the control group(s), pre-implantation surface roughness was positively correlated with 4 week rabbit BMP2 (0.997; p=0.0467) and negatively correlated with 8 week pins (−0.896; p=0.0398).

The purpose of this example was to examine how patterning of bone substrates was tolerated in an in vivo pre-clinical animal model after having been proven to be successful in in vitro models. In prior in vitro studies, laser patterning was performed on a flat substrate, and this study demonstrates that patterning can take place on curved surfaces. Protein content in pin tissue was greater than rabbit tissue for both control and treatment groups at 4 and 8 weeks post-implantation. This is indicative of the healing response, particularly that the host is incorporating/remodeling the implant. Furthermore, the 8 week time point was significantly greater than the 4 week time point for many proteins in both pin and rabbit. This response was more pronounced in the treatment (patterned) group than the control group.

Accordingly, this example demonstrates that human allograft bone pins are well tolerated by the host subject, regardless of patterning. Moreover, patterned implants produced a significant and positive response regarding healing speed and bone remodeling, especially at later time points.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A bone product comprising bone having at least one patterned surface, the patterned surface comprising a plurality of pits, each of the plurality of pits having a diameter of from about 5 μm to about 15 μm and a depth of approximately half of the diameter.

2. The bone product according to claim 1, wherein the plurality of pits have a pitch of between about 1.5 times and about 2.5 times the diameter.

3. The bone product according to claim 1, wherein the bone comprises cortical bone.

4. The bone product according to claim 1, wherein the bone comprises cancellous bone.

5. The bone product according to claim 1, wherein each of the plurality of pits has a diameter of from about 7 μm to about 12 μm.

6. The bone product according to claim 5, wherein each of the plurality of pits has a diameter of about 10 μm.

7. The bone product according to claim 1, wherein the patterned surface comprises a curved surface.

8. A bone product comprising cortical bone having at least one patterned surface, the patterned surface comprising a plurality of pits, each of the plurality of pits having a diameter from about 5 μm to about 15 μm and a pitch of about 1.5 times to about 2.5 times the diameter of each of the plurality of pits.

9. The bone product according to claim 8, wherein each of the plurality of pits has a diameter of from about 7 μm to about 12 μm.

10. The bone product according to claim 9, wherein each of the plurality of pits has a depth of approximately half the diameter.

11. The bone product according to claim 8, wherein the bone product is seeded with stem cells.

12. The bone product according to claim 11, wherein the stem cells comprise adipose-derived stem cells.

13. The bone product according to claim 11, wherein the stem cells comprise bone marrow-derived stem cells.

14. An allograft bone comprising at least one surface modification, the surface modification comprising a plurality of pits, each of the plurality of pits having a diameter of from about 5 μm to about 15 μm.

15. The allograft bone according to claim 14, wherein each of the plurality of pits has a depth of approximately half of the diameter.

16. The allograft bone according to claim 15, wherein the plurality of pits have a pitch of about 20 μm.

17. The allograft bone according to claim 16, wherein the bone comprises cortical bone.

18. The allograft bone according to claim 16, wherein the bone comprises cancellous bone.

19. The allograft bone according to claim 16, wherein each of the plurality of pits is substantially hemispherical in shape.

20. The allograft bone according to claim 19, wherein each of the plurality of pits has a diameter of from about 7 μm to about 12 μm.
